(19) United States
(12) Patent Application Publication
(10) Pub. No.: US 2014/0099636 A1
(43) Pub. Date: Apr. 10, 2014

(54) FIELD-BASED qPCR MICROBIAL MONITORING

(71) Applicant: Baker Hughes Incorporated, Houston, TX (US)

(72) Inventor: Crystal Lee, Sugar Land, TX (US)

(73) Assignee: Baker Hughes Incorporated, Houston, TX (US)

(21) Appl. No.: 14/045,366

(22) Filed: Oct. 3, 2013

Related U.S. Application Data
(60) Provisional application No. 61/711,790, filed on Oct. 10, 2012.

(51) Int. Cl. C12Q 1/68

(52) U.S. Cl. CPC C12Q 1/689
USPC 435/6.11; 435/6.12

(57) ABSTRACT

DNA/RNA monitoring of microbes at an oil field to determine the presence and activity of harmful microbes is accomplished with a portable qPCR (quantitative polymerase chain reaction) machine. This permits the monitoring to occur on-site in the field and reduces the variability that may occur from the transportation of samples.

### CROSS-REFERENCE TO RELATED APPLICATION

This application claims the benefit of provisional patent application 61/711,790 filed Oct. 10, 2012, incorporated by reference herein in its entirety.

### TECHNICAL FIELD

The present invention relates to methods and apparatus for detecting and quantifying microbial processes in the oilfield, and more particularly relates, in one non-limiting embodiment, to methods and apparatus for detecting and quantifying microbial activity and processes in oilfield operations using PCR/qPCR (quantitative polymerase chain reaction) methods.

### BACKGROUND

Biofouling, caused by the attachment and growth of prokaryotes in the oilfield, as well as other industries, leads to accelerated microbiologically influenced corrosion (MIC) rates, emulsion problems, the plugging of filters, and hydrogen sulfide (H₂S) production, which is hazardous, corrosive, generates FeS (ferrous sulfide or iron (II) sulfide) scale, and eventually causes souring of the formation. Biofouling in the oilfield usually involves the formation of biofilms, which are structured communities of microorganisms encapsulated within a polymeric matrix developed by prokaryotes. Biofilm adheres to an inert surface and often consists of mixed species of prokaryotes. MIS (microbiologically influenced souring) and planktonic microbes (which are non-biofilm-forming) may also cause problems in the oilfield. Current techniques used to quantify oilfield microbes focus on microscopy to physically count all the bacteria (living and dead, with no distinction between them) in a portion of a sample, or culturing the sample in “bug bottles” to quantify specific groups of bacteria. These methods have been employed in the oilfield for decades and have proven effective in discerning and enumerating relative bacterial populations. However, these methodologies, due to inherent limitations, do not satisfy the needs of a growing industry with respect to expediency, accuracy and correlation to microbial problem. For example, current practices for monitoring MIC depend on either directly quantifying all bacterial cells in a sample via microscopy or culturing specific groups of prokaryotes, usually sulfate-reducing prokaryotes (SRP), nitrate-reducing bacteria (NRB) or methanogens. However, MIC is caused by a community of bacteria in a biofilm and there is no scientific correlation between numbers and types of cells and localized corrosion. Microscopy, although reasonably accurate for counting bacteria, only covers a certain dilution range (greater than 10⁴ bacteria per ml) and cannot distinguish between live vs. dead bacteria, nor can it identify groups of bacteria or their activity. Furthermore, culture methods only recover an estimated 0.1-10% of the original population and in some cases fail to detect various organisms in the sample as in the case of thermophilic (high temperature) prokaryotes.

In particular, current techniques produce very inconsistent results in determining the biological activity of the organisms present. There are common reports from the field that bacteria and/or archaea counts from current methods do not correlate with measured rates of H₂S production or corrosion.

Additionally, while DNA/RNA monitoring of prokaryotes in an oilfield may be used to determine the presence and activity of harmful microbes, this technology is currently implemented by transporting field samples to a remote laboratory. Sample variability may occur during the storage and transport of samples, including degradation and/or growth of the microbes.

Therefore, new techniques need to be developed that detect and quantify oilfield microbial processes and mechanisms more reliably and quickly. It would thus be desirable if new methods, techniques and/or apparatus or systems would be devised to detect and quantify microbial processes in the oilfield.

### SUMMARY

There is provided, in one non-limiting form, a method of monitoring microbes at an oilfield comprising extracting a microbial sample onto a clean surface at an oilfield, where the sample is selected from the group consisting of DNA, RNA or a combination thereof; amplifying the microbial sample with PCR reagents at the oilfield; and enumerating and identifying the DNA using PCR or qPCR in the microbial sample at the oilfield.

In another non-restrictive version, there is provided a method of quantitative detection of microbial processes in an oilfield which comprises extracting a microbial field sample believed to contain nucleotide sequences onto a clean surface at an oilfield and amplifying the field sample nucleotide sequences with PCR reagents at the oilfield to give quantifiable amplified field sample nucleotide sequences. The method additionally comprises quantifying field sample nucleotide sequences concurrently with a molecular label that provides a detectable signal. The method further includes enumerating and identifying the nucleotide sequences in the microbial field sample at the oilfield with PCR/qPCR. The PCR/qPCR comprises using primers and/or probes that target specific DNA sequences that hybridize to field sample nucleotide sequences previously identified with microbial metabolic activity. The microbial metabolic activity includes, but is not necessarily limited to cellular respiration; thermophilic bacteria/archaea activity; production of a compound selected from the group consisting of an organic acid, a surfactant, a polymer, a solvent, a gas, and combinations thereof; degradation of a compound selected from the group consisting of organic acids, petroleum hydrocarbons, xenobiotics and combinations thereof; enzymatic processes involved in the enhancement of the recovery or refinement of crude oil; and combinations thereof. The method further involves amplification of the target nucleotide sequences to generate a quantifiable detectable signal, and analyzing the detectable signal to quantify the microbial number or metabolic activity.

In more detail, there are primers to direct the enzyme to amplify the region of interest (ROI). The probe used would be a short nucleotide sequence that is complementary to a central region in the ROI, and the probe will hybridize to the ROI and be degraded by the exonuclease action of the DNA polymerase. The probe has a quencher and fluorescent tag which are both released during amplification. The release of this fluorescent tag (in one non-limiting example, fluorescin) from proximity of the quencher allows for the fluorescence to occur. Increased fluorescence detected will indicate more amplification of the ROI.

As used herein, the term “oilfield” includes any field or location involved in the exploration and/or production of crude oil, natural gas and/or other hydrocarbon.

### DETAILED DESCRIPTION

All processes within an organism are fundamentally controlled by genes. An organism constantly senses and responds to environmental stimuli (presence of nutrients, temperature extremes, dehydration, etc.) by turning on or off genes. When genes are “turned on”, mechanisms in the bacteria cell produce molecules called ribonucleic acid (RNA) which translates the DNA code of the gene into a protein. The protein helps implement or regulate a specific microbial process. The amount of protein is proportional to the number of RNAs produced and hence to the relative activity of the gene. The more a gene is activated, the more RNA will be produced resulting in the increased activity of a microbial process. Therefore, if the genes have been identified, a microbial process can be quantified by quantifying the amount of its specific RNA within the cell.

Recent advances in biotechnology have allowed the development of PCR/qPCR assays that are customizable. This methodology involves designing primers and/or probes to specific DNA sequences to target genes of interest. For instance PCR/qPCR can then be used to screen the genetic sequences of a given field sample. In a non-limiting example, a PCR/qPCR assay detecting enzymes involved in H₂S generation may be used to quantify the SRP metabolic activity for the production of H₂S in field samples. The metabolic process for the conversion of sulfate to sulfide in SRPs has been elucidated and the genetic sequences involved have been characterized. GenBank, an open access, annotated genetic sequence database with over 100 million searchable genetic sequences, may be used to select target nucleotide sequences or probes for SRP metabolic activity.

Selected sequences to be analyzed for may be submitted to a company that has the technology to manufacture a PCR/qPCR chip that includes the primers and/or probes for microbial analysis. Such companies include, but are not limited to InstantLabs LLC, AquaBioChip LLC, Affymetrix, Inc. or Eppendorf International.

It may be noted that field sample RNA may directly hybridize with a target sequence or it may be converted into cDNA (copy DNA which is more stable) to be hybridized with a target sequence. The final form of the H₂S biochip would be able to quantify field samples within a relatively short period of time, for instance, within 36 hours of receipt for theoretically any type of SRP on-site at an oilfield; alternatively within 40 hours or less, or within 24 hours or less, or even 8 hours or less. In one non-limiting embodiment the lower threshold of this time period is 0.5 hours, or alternatively, 1 hour. Other such biochip developments may include PCR/qPCRs to detect microbial activity involved in corrosion, microbial enhanced oil recovery (MEOR) and also for thermophilic bacteria or other groups of oilfield bacteria that are problematic to detect by conventional methods.

As noted, currently field samples are physically transported to a laboratory for analysis. Using a portable qPCR (quantitative polymerase chain reaction) machine would allow the monitoring to occur on-site, at the oilfield thereby reducing variability that may occur from the transportation of the samples. Difficulties that may occur over the time and exposure to varied environments of this storage and transportation may include, but not necessarily be limited to, sample degradation, bacterial growth (thereby exaggerating the perceived amount of bacteria at the oilfield sample site), and/or change in bacterial population distribution.

For DNA-based detection, a liquid or solid sample to be analyzed may be extracted at the oilfield on a cleaned surface, and then samples may be loaded onto a reaction chamber and amplified with PCR/qPCR reagents to enumerate bacterial DNA. For RNA-based detection, a liquid or solid sample to be analyzed may be extracted in the field onto a clean surface, converted to cDNA with reverse transcriptase, then loaded onto a reaction chamber and amplified with PCR/qPCR reagents.

The method of monitoring bacteria at an oilfield herein may be accomplished between about 0.5 independently to about 48 hours; alternatively from about 4 independently to about 40 hours, and in another non-limiting embodiment from about 8 independently to about 24 hours. The method may be accomplished within about 150 miles of the oilfield; alternatively within about 100 miles of the oilfield, in another non-limiting embodiment about 50 miles of the oilfield. In another non-restrictive embodiment, “at the oilfield” herein should be understood as including a field lab that is within a 12 hour round-trip travel time to the well site; alternatively within a 10 hour round-trip, and in another non-limiting version within an 8 hour round-trip. Further, sample degradation is reduced (or stated another way, sample integrity is increased) with the method described herein as compared with conducting a similar method where amplifying the bacterial sample and identifying the DNA in the bacterial sample are performed at a laboratory remote from the oilfield as is conventionally done.

In one non-limiting embodiment, identifying the microbial DNA is accomplished using molecular labels where the molecular label is fluorescent. Laser scanners, UV-vis components or charge coupled device (CCD) sensors (or other suitable devices) may be used to read the fluorescent intensities (emissions spectra) in reaction chambers and statistical software applications may be applied or used for quantification. The system would also catalog the features exhibiting fluorescence with the specific DNA nucleotide sequence corresponding to a specific gene on a given feature. Total procedural time from sample preparation to analysis may be relatively short; in a non-limiting embodiment approximately 40 hours; alternatively 24 hours or less.

In one non-limiting embodiment, identifying the microbial DNA involves identifying microbial metabolic activity including, but not necessarily limited to, cellular respiration (including, but not necessarily limited to iron reduction and nitrate reduction), thermophilic bacteria/archaea activity, production of a compound selected from the group consisting of an organic acid, a surfactant, a polymer, a solvent, a gas, and combinations thereof, degradation of a compound selected from the group consisting of organic acids, petroleum hydrocarbons, xenobiotics and combinations thereof, enzymatic processes involved in the enhancement of the recovery or refinement of crude oil, and combinations thereof. More specifically within the compounds whose production may be identified, the organic acid may include, but is not necessarily limited to, acetic acid, propionic acid, butyric acid and combinations thereof. The surfactant may include, but is not necessarily limited to, peptides, saccharides, lipids, and combinations thereof. The gas may include, but not necessarily be limited to, methane (CH₄), hydrogen (H₂), carbon dioxide (CO₂), hydrogen sulfide (H₂S), and combinations thereof. The solvents may include, but are not necessarily limited to, acetone, ethanol, butanol, aldehydes and combinations thereof. Finally, the polymers may include, but are not necessarily limited to, exopolysaccharides such as alginate, xanthan gum, dextran, and combinations thereof.

In the foregoing specification, it will be evident that various modifications and changes may be made thereto without departing from the broader spirit or scope of the invention as set forth in the appended claims. Accordingly, the specification is to be regarded in an illustrative rather than a restrictive sense. For example, target nucleotide sequences to detect genes in the oilfield on-site other than those mentioned, but not specifically identified or tried in a particular method or composition, are anticipated to be within the scope of this invention. Further, it will be appreciated that more than one field sample nucleotide sequence may be addressed with a single PCR/qPCR assay, in a non-limiting instance a gas such as H₂S and a particular organic acid with the purpose of addressing more than one corrosion mechanism. Additionally, portable qPCR machines and PCR/qPCRs that fall within the methods and apparatus herein, but that are made by different methods and processing that those specifically outlined are also within the scope of the invention herein.

The present invention may suitably comprise, consist or consist essentially of the elements disclosed and may be practiced in the absence of an element not disclosed.

The words “comprising” and “comprises” as used throughout the claims is to interpreted “including but not limited to”. For instance, the method of monitoring microbes at an oilfield may consist of or consist essentially of extracting a microbial sample onto a clean surface at an oilfield, where the microbial sample is selected from the group consisting of DNA, RNA or a combination thereof; amplifying the microbial sample with PCR reagents at the oilfield; and enumerating and identifying the DNA in the microbial sample at the oilfield.

In another non-limiting embodiment, a method of quantitative detection of microbial processes in an oilfield may consist of or consist essentially of extracting a microbial field sample believed to contain nucleotide sequences onto a clean surface at an oilfield; amplifying the field sample nucleotide sequences with PCR reagents at the oilfield to give quantifiable amplified field sample nucleotide sequences; quantifying the amplified field sample nucleotide sequences with a molecular label that provides a detectable signal; enumerating and identifying the nucleotide sequences in the microbial field sample at the oilfield with a PCR/qPCR as described herein; amplifying the target nucleotide sequences to generate a quantifiable detectable signal; and analyzing the detectable signal to quantify the microbial number or metabolic activity.

What is claimed is:

1. A method of monitoring microbes at an oilfield comprising:
extracting a microbial sample onto a clean surface at an oilfield, where the sample is selected from the group consisting of DNA, RNA or a combination thereof;
amplifying the microbial sample with PCR reagents at the oilfield; and
enumerating and identifying the DNA in the microbial sample at the oilfield.

2. The method of claim 1 where in the case the microbial sample is RNA, after the microbial sample is extracted onto a clean surface, the RNA is converted to cDNA with reverse transcriptase prior to amplifying the microbial sample with PCR reagents.

3. The method of claim 1 where identifying the microbial DNA comprises identifying microbial metabolic activity selected from the group consisting of:
cellular respiration;
thermophilic bacteria/archaea activity;
production of a compound selected from the group consisting of an organic acid, a surfactant, a polymer, a solvent, a gas, and combinations thereof;
degradation of a compound selected from the group consisting of organic acids, petroleum hydrocarbons, xenobiotics and combinations thereof;
enzymatic processes involved in the enhancement of the recovery or refinement of crude oil; and
combinations thereof.

4. The method of claim 3 where:
the organic acid is selected from the group consisting of acetic acid, propionic acid, butyric acid and combinations thereof;
the surfactant is selected from the group consisting of peptides, saccharides, lipids, and combinations thereof;
the gas is selected from the group consisting of CH₄, H₂, CO₂, H₂S, and combinations thereof;
the solvents are selected from the group consisting of acetone, ethanol, butanol, aldehydes and combinations thereof; and
the polymers are exopolysaccharides selected from the group consisting of alginate, xanthan gum, dextran, and combinations thereof.

5. The method of claim 1 where identifying of microbial DNA is accomplished using molecular labels where the molecular label is fluorescent.

6. The method of claim 1 where the method is accomplished between about 0.5 to about 40 hours.

7. The method of claim 1 where the method is accomplished within 150 miles of the oilfield.

8. The method of claim 1 where sample degradation is reduced as compared with conducting a similar method where amplifying the microbial sample and identifying the DNA in the microbial sample are performed at a laboratory remote from the oilfield.

9. The method of claim 1 further comprising practicing the method using a portable qPCR machine.

10. A method of quantitative detection of microbial processes in an oilfield comprising:
extracting a microbial field sample believed to contain nucleotide sequences onto a clean surface at an oilfield;
amplifying the field sample nucleotide sequences with PCR reagents at the oilfield to give quantifiable amplified field sample nucleotide sequences;
quantifying the amplified field sample nucleotide sequences with a molecular label that provides a detectable signal;
enumerating and identifying the nucleotide sequences in the microbial field sample at the oilfield with a PCR/qPCR that comprises:
using primers and/or probes that target specific DNA sequences that hybridize to field sample nucleotide sequences previously identified with microbial metabolic activity selected from the group consisting of:
cellular respiration;
thermophilic bacteria/archaea activity;
production of a compound selected from the group consisting of an organic acid, a surfactant, a polymer, a solvent, a gas, and combinations thereof;
degradation of a compound selected from the group consisting of organic acids, petroleum hydrocarbons, xenobiotics and combinations thereof;
enzymatic processes involved in the enhancement of the recovery or refinement of crude oil; and
combinations thereof;
amplifying the target nucleotide sequences to generate a quantifiable detectable signal; and
analyzing the detectable signal to quantify the microbial number or metabolic activity.

11. The method of claim 10 where:
the organic acid is selected from the group consisting of acetic acid, propionic acid, butyric acid and combinations thereof;
the surfactant is selected from the group consisting of peptides, saccharides, lipids, and combinations thereof;
the gas is selected from the group consisting of CH₄, H₂, CO₂, H₂S,and combinations thereof;
the solvents are selected from the group consisting of acetone, ethanol, butanol, aldehyde and combinations thereof; and
the polymers are exopolysaccharides selected from the group consisting of alginate, xanthan gum, dextran and combinations thereof.

12. The method of claim 10 where the quantifiable detectable signal is fluorescence.

13. The method of claim 10 where the method is accomplished between about 0.5 to about 40 hours.

14. The method of claim 10 where the method is accomplished within 150 miles of the oilfield.

15. The method of claim 10 where sample cleanliness is improved by as compared with conducting a similar method where amplifying the microbial sample and identifying the DNA in the microbial sample are performed at a laboratory remote from the oilfield.

16. The method of claim 10 further comprising practicing the method using a portable qPCR machine.

17. A method of quantitative detection of microbial processes in an oilfield comprising:
extracting a microbial field sample believed to contain nucleotide sequences onto a clean surface at an oilfield;
amplifying the field sample nucleotide sequences with PCR reagents at the oilfield to give quantifiable amplified field sample nucleotide sequences;
quantifying the amplified field sample nucleotide sequences with a molecular label that provides a detectable signal;
enumerating and identifying the nucleotide sequences in the microbial field sample at the oilfield with a PCR/qPCR that comprises:
using primers and/or probes that target specific DNA sequences that hybridize to field sample nucleotide sequences previously identified with microbial metabolic activity selected from the group consisting of:
cellular respiration;
thermophilic bacteria/archaea activity;
production of a compound selected from the group consisting of an organic acid, a surfactant, a polymer, a solvent, a gas, and combinations thereof;
degradation of a compound selected from the group consisting of organic acids, petroleum hydrocarbons, xenobiotics and combinations thereof;
enzymatic processes involved in the enhancement of the recovery or refinement of crude oil; and
combinations thereof;
amplifying the target nucleotide sequences to generate a quantifiable detectable signal that is fluorescence.; and
analyzing the detectable signal to quantify the microbial number or metabolic activity;
further comprising practicing the method using a portable qPCR machine.

18. The method of claim 17 where:
the organic acid is selected from the group consisting of acetic acid, propionic acid, butyric acid and combinations thereof;
the surfactant is selected from the group consisting of peptides, saccharides, lipids, and combinations thereof;
the gas is selected from the group consisting of CH₄, H₂, CO₂, H₂S,and combinations thereof;
the solvents are selected from the group consisting of acetone, ethanol, butanol, aldehyde and combinations thereof; and
the polymers are exopolysaccharides selected from the group consisting of alginate, xanthan gum, dextran and combinations thereof.

19. The method of claim 17 where the method is accomplished between about 0.5 to about 40 hours.

20. The method of claim 17 where sample cleanliness is improved by as compared with conducting a similar method where amplifying the microbial sample and identifying the DNA in the microbial sample are performed at a laboratory remote from the oilfield.
